SEQUENCE LISTING (9 sequences)

SEQ ID NO: 1 - RNA, 23 nt, Artificial Sequence
  gRNA sequence
gggcccacggcacggaacagcgg

SEQ ID NO: 2 - DNA, 1326 bp, Salmo salar
gaaagttgctactttttcgagacctaggataatggaggagcgttcaagtcaggtgttgaacccggagcgactgaaggcgctggagatgtggctgcaggagactgacgtcaaactgacccaggtcaatggccagaggaaatatggaggtccacctgatgactggcttggcgccccccctgggccgggctgtgaggtgttcatcagccagatcccgcgggatgtctttgaggaccagctgattccgctgttccgtgccgtgggccctctctgggagttccgcctcatgatgaacttcagcggacagaaccgtggctttgcctacgccaagtacgacagccctgcctcggccgctgccgccatccgctcgttgcatggccgtgccctcgagtcaggggcacgcctcggtgtacggcgcagcacggagaaacgtcagctctgtcttggggagctgcccaccagcacaaggagggagcaactgctgcaggtgctgctggacttctctgagggggtagagggcgtgtccctgagagcagggcctggggaacaggggatgtctgcagtggtggtctatgcctcccaccatgcagcttccatggccaagaaggtgctgattgaagcctttaaaaaacgcttcgggctggccatcactttgaagtggcagtcctcttctaggcccaagcacgaagagcctcccagaccctccaaaacccctccttcctctcctcccaaacctcctcgctgctccctcctggacagcccccggcctcccctgcacctcgcccagcgtcagctccctgccttctcccgggctgtgagggcgccctctcccatggtgcacgctgctcctgaatcccccaggggggcgaccatggtgcctcctgtggatgcagcagccctgctccagggtgtgtgtgaggtgtacgggcaggggaagcccctctatgacctgcagtaccgccacatggggcctgacgggttcctgtgcttcagctaccgggtgtatgtgccggggctggccacacccttcactgggatggtgcagactctgcccggccccacccctggagccatacaggaagaggctcgcagagctacagcccagcaggtcctcagcgctctgtacagggcctgatggtgttgaagcacagatcccctactttgttttaattatgaaaatacttaaatgttttgcactcttttatatttagtaagtagatgcatgattttacttttttttttgaaccacttttgcatgtttctgcaccatttaattgtttctcattataataaaatgagatttgtcaaaaaaaaaaaaaaaaaaaaaaa

SEQ ID NO: 3 - DNA, 31 bp, Salmo salar
cggaacagcggaatcagctggtcctcaaaga

SEQ ID NO: 4 - DNA, 11 bp, Salmo salar
cggaacagcgg

SEQ ID NO: 5 - DNA, 23 bp, Salmo salar
cggaatcagctgggcctcaaaga

SEQ ID NO: 6 - DNA, 23 bp, Salmo salar
cggaatcagctggtcctcctgga

SEQ ID NO: 7 - DNA, 24 bp, Salmo salar
cggaatcccgcaggcccccgaaaa

SEQ ID NO: 8 - DNA, 23 bp, Salmo salar
cggaatcagctggtcctcaaaga

SEQ ID NO: 9 - DNA, 24 bp, Salmo salar
cggaactccggaggcccttggaca

(19) United States
(12) Patent Application Publication
(10) Pub. No.: US 2023/0134819 A1
(43) Pub. Date: May 4, 2023

(54) MODIFIED SALMON WHICH PRODUCE STERILE OFFSPRING

(71) Applicant: VESTLANDETS INNOVASJONSSELSKAP AS, Bergen (NO)

(72) Inventors: Anna TROEDSSON-WARGELIUS, Paradis (NO); Rolf Brudvik EDVARDSEN, Bergen (NO)

(21) Appl. No.: 17/916,114

(22) PCT Filed: Apr. 1, 2021
(86) PCT No.: PCT/EP2021/058629

§ 371 Date: Sep. 30, 2022

(30) Foreign Application Priority Data
Apr. 2, 2020 (GB) 2004870.8

(51) Int. Cl. A01K 67/027; C07K 14/46; C12N 15/90; C12N 15/113

(52) U.S. Cl. CPC A01K 67/0276; C07K 14/461; C12N 15/90; C12N 15/113; A01K 2217/077; A01K 2227/40; A01K 2267/02; C12N 2310/20; C12N 2510/00

(57) ABSTRACT

The present invention relates, inter alia, to processes for making modified fish zygotes or early-stage fish embryos (particularly salmon zygotes and salmon embryos). The invention also provides fish zygotes, fish embryos, juvenile fish, mature fish and sterile fish which are produced by the processes of the invention.

The present invention relates, inter alia, to processes for making modified fish zygotes or early-stage fish embryos (particularly salmon zygotes and salmon embryos). The invention also provides fish zygotes, fish embryos, juvenile fish, mature fish and sterile fish which are produced by the processes of the invention.

The salmon aquaculture industry is a major driving force for novel biotechnological applications. Such biotechnology can be used to solve the major aquaculture bottlenecks that currently limit a sustainable expansion of the salmon farming industry [1] both at sea and in closed systems.

One major bottleneck is the genetic impact of escaped farmed salmon on wild populations and the undesirable intermixing of the genes from wild and farmed salmon.

There are three main reasons for interest in inhibiting sexual activity in farmed fish: management difficulties (includes the problem with escaped fish), to reduce aggressive and sexual behaviour and to improve growth, meat and carcass quality (includes the problem with unwanted maturity in fish).

Inhibiting sexual activity in mammals can be done surgically, but also with more sophisticated methods such as immunisation against GnRH which causes a temporary castration-like effect in, for example, boars and horses. In bulls, the testis is surgically removed to ensure more meat and better quality (e.g. Reproductive Technologies in Farmed Animals, 2^{nd} edition, 2017). However, these methods do not apply well to fish because surgical removal would create an overwhelming amount of work with low survival considering the large number of animals and the internal location of testis in fish.

Short term castration through hormone vaccination would not work very well either, since it only temporarily delays puberty, but does not inhibit reproduction [2].

A new approach to induce sterility is by ablating germ cells. This has recently been shown in zebrafish, where embryos were bathed in a solution which contained vivo-morpholinos which blocked an mRNA encoding a protein essential for development of germ cells [3]. This bath technique may not be general to all fish since the protein may have diverging functions between fish species; also, every egg batch must be treated which will be laborious and expensive. In addition, the solution may be toxic to the embryos and it may not always be 100% effective [4], resulting in similar problems that are currently found in triploid fish production [5].

The only method used in commercial-scale production of sterile salmon is triploidisation. However, triploid salmon are more sensitive to suboptimal rearing environments. For example, vertebral deformities and cataracts are observed more frequently in triploids than in diploids [5]. These negative effects have led to concerns regarding fish welfare in commercially-farmed triploid salmon and the Norwegian Food authorities (see https://www.mattilsynet.no/language/english/) has been critical of this production method. In addition, the production of triploids is often incomplete and 5-20% percent may be diploid. If these fish escape, the problem with genetic introgression will remain.

Whilst the farming of sterile animals overcomes the issue of how to prevent the cross-breeding of domesticated and wild animals, biotechnological methods of producing such sterile animals can be time-consuming and expensive [6, 7]. In addition, the welfare and other relevant production traits may be affected such as lower welfare, disease resistance and mating behaviour, as in triploid sterile farmed salmon [5].

The invention presented here describes a method that ensures broodstock fish produce 100% sterile offspring. This approach solves the problems with genetic introgression, precocious maturation and support the breeding industry in protecting their genetic innovations thus representing a significant commercial potential.

The invention is based on the concept of producing fertile broodstock from F0 fish which have been modified to lack germ cells by reducing or eliminating functional expression of a gene involved in germ cell survival, wherein the germ cell survival factor gene is a piwil gene or a piwi gene (e.g. piwil1). Primordial germ cells may be rescued in F1 fish zygotes by adding a normal variant of the mutated germ cell survival factor gene, either as mRNA or a protein, during the early phase of germ cell development, in order to produce further broodstock fish.

The invention provides fertile broodstock (F1) fish which can produce sterile (F2) fish for farming, e.g. for food production. This invention helps companies to preserve their genetic brand, which may also include other beneficial genetic modifications such as resistance to diseases (salmon lice, etc.).

Expression of the piwil1 gene has previously been described in Atlantic salmon [10]. However, a number of papers have suggested a juvenille function for piwi in germ cell survival [19, 20].

A process to make non-sterile fish has now been found wherein the fish lack the Piwil1 protein in the adult germ cells. It is an object of the invention therefore to provide a process for producing a modified fish zygote or fish embryo, which can be grown to produce a first generation (F1) of fish which, whilst being non-sterile themselves, produce viable gametes which produce sterile (F2, second generation) offspring. It is also object of the invention to provide such first-generation fish and such second-generation fish. It is also the object of the invention to establish a stable broodstock to which additional sustainable genetic traits can be added.

In one embodiment, the invention provides a process for producing a modified fish zygote or modified early-stage fish embryo, the process comprising the step:
(a) introducing protein or mRNA encoded by a germ cell survival factor gene, wherein the germ cell survival factor gene is a piwil gene or a piwi gene (e.g. piwil1) into a fish zygote or one or more cells of an early-stage fish embryo,

wherein the genome of the fish zygote or the genomes of the one or more cells of the early-stage fish embryo comprises one or more mutations which render one or more copies of the endogenous germ cell survival factor gene or its gene product non-functional.

Preferably, both (if the genome is diploid) or all (if the genome is polyploid) copies of the endogenous germ cell survival factor gene or its gene product are (have been) rendered non-functional in the fish zygote.

Preferably, all copies of the endogenous germ cell survival factor gene or its gene product are (have been) rendered non-functional in all cells of the early-stage fish embryo.

The invention also provides a process for producing a modified fish zygote or modified early-stage fish embryo, the process comprising the step:
(a) modifying the genome of a fish zygote or the genome of one or more cells of an early-stage fish embryo to eliminate functional expression of a germ cell survival factor gene, wherein the germ cell survival factor gene is a piwil gene or a piwi gene (e.g. piwil1),

optionally wherein the fish zygote or the cells of the early-stage fish embryo are ones which comprise a non-wild-type amount of the germ cell survival factor RNA or protein.

Preferably, functional expression from both (if the genome is diploid) or all (if the genome is polyploid) copies of the germ cell survival factor gene is eliminated in the fish zygote in Step (a). Preferably, functional expression from all copies of the germ cell survival factor gene is eliminated in all of the cells of the early-stage fish embryo in Step (a).

The invention also provides a process for producing a modified fish zygote or a modified early-stage fish embryo, the process comprising the steps:
(a) modifying the genome of a fish zygote or one or more cells of an early-stage fish embryo to eliminate functional expression of a germ cell survival factor gene, wherein the germ cell survival factor gene is a piwil gene or a piwi gene (e.g. piwil1); and optionally
(b) introducing functional protein or RNA encoded by the germ cell survival factor gene into the fish zygote or the one or more cells of the early-stage fish embryo.

Preferably, the functional expression of both (if the genome is diploid) or all (if the genome is polyploid) copies of the germ cell survival factor gene are eliminated in the fish zygote in Step (a). Preferably, the functional expression of all copies of the germ cell survival factor gene are eliminated in all of the cells of the early-stage fish embryo in Step (a).

The invention also provides a modified fish zygote or modified early-stage fish embryo, wherein the fish zygote or one or more cells of the early-stage fish embyro comprises a non-wild-type amount of a germ cell survival factor polypeptide or RNA, wherein the polypeptide is a PIWIL or PIWI polypeptide or the RNA is a piwil or piwi RNA.

In some embodiments, the modified fish zygote or one or more cells of the early-stage fish embryo additionally comprises a CRISPR enzyme (e.g. Cas9) and/or a gRNA comprising a piwil or piwi gene-targeting sequence.

The invention also provides a modified fish zygote, wherein the zygote comprises a non-wild-type amount of mRNA encoding a germ cell survival factor, wherein the germ cell survival factor mRNA is a piwil mRNA or a piwi mRNA (e.g. piwil1 mRNA), and wherein the fish zygote does not comprises an anti-piwil or anti-piwi morpholino.

In some embodiments, the genome of the fish zygote (e.g. 2^{nd} generation and subsequent generations of the broodstock) is not capable of functional or viable expression of the germ cell survival factor gene. In other embodiments (e.g. in broodstock production), the fish zygote expresses a non-functional germ cell survival factor mRNA or protein.

The invention also provides a process for producing a broodstock fish, the process comprising the steps:
(a)(i) culturing a fish zygote or early-stage fish embryo of the invention, or
(a)(ii) producing a modified fish zygote or early-stage fish embryo by a process for producing a modified fish zygote or early-stage fish embryo of the invention and culturing the fish zygote or early-stage fish embryo to produce a cultured fish; and
(b) growing the cultured fish to produce a juvenile broodstock fish, and optionally
(c) growing the juvenile broodstock fish to produce a sexually-mature broodstock fish.

The invention also provides a juvenile or sexually-mature fish:
(a) whose genome comprises one or more (preferably 3-20) mutations in a germ cell survival factor gene, wherein the germ cell survival factor gene is a piwil gene or a piwi gene (e.g. piwil1), wherein the one or more mutations render all copies of the germ cell survival factor gene or gene product non-functional; and
(b) which has gonads which are capable of producing viable sperm or eggs.

The invention also provides sperm or eggs from a sexually-mature fish of the invention.

The invention also provides a fish zygote (a) wherein the zygote does not comprise any functional RNA encoded by a germ cell survival factor gene, wherein the germ cell survival factor gene is a piwil gene or a piwi gene (e.g. piwil1). Preferably, the genome of the zygote comprises one or more (preferably 3-20) mutations which render one or more or all copies of the germ cell survival factor gene non-functional.

The invention also provides a fish zygote (a) wherein the zygote does not comprise a functional protein encoded by a germ cell survival factor gene, wherein the germ cell survival factor gene is a piwil gene or a piwi gene (e.g. piwil1). Preferably, the genome of the zygote comprises one or more (preferably 3-20) mutations which render one or more or all copies of the germ cell survival factor gene non-functional.

The invention also provides a process for producing a sterile fish, the process comprising the steps:
(a) culturing a fish zygote of the invention to produce a cultured sterile fish; and
(b) growing the cultured fish to produce a juvenile sterile fish; and optionally
(c) growing the juvenile sterile fish to produce an adult sterile fish.

The invention also provides a sterile fish which has been produced by the above process.

In yet another embodiment, the invention provides a sterile fish (preferably a salmon):
(a) whose genome comprises one or more (preferably 1-2) mutations which render one or more or all copies of a germ cell survival factor gene, wherein the germ cell survival factor gene is a piwil gene or a piwi gene (e.g. piwil1) non-functional; and
(b) wherein the physiological and/or anatomical features of the fish are characteristic of a fish that has developed from a zygote which was lacking in maternally-derived mRNA for the germ cell survival factor gene.

Preferably, the zygote contained no maternally-derived mRNA for the germ cell survival factor gene.

In yet another embodiment, the invention provides a process for producing a modified fish zygote or modified early-stage fish embryo, the process comprising the step:
(a) introducing protein or RNA encoded by a germ cell survival factor gene wherein the germ cell survival factor gene is a piwil gene or a piwi gene (e.g. piwil1) into a fish zygote or early-stage fish embryo,

wherein the genome of the fish zygote or early-stage fish embryo comprises one or more (preferably 1-2) mutations which render all copies of the germ cell survival factor gene non-functional.

The fish is preferably one which is or can be commercially harvested for food or for recreational purposes. The term “fish” includes salmon, trout (e.g. brown trout and rainbow trout), carp, tilapia, catfish, sea bass, sturgeon, halibut, cod and seabream. Preferably, the fish is from the family Salmonidae. The subfamily Salmoninae includes: Brachymystax—lenoks; Eosalmo (Eocene); Hucho; Oncorhynchus—Pacific salmon and trout; Parahucho—Sakhalin taimen; Salmo—Atlantic salmon and trout; Salvelinus—Char and trout (e.g. brook trout, lake trout); and Salvethymus—Long-finned char. The genus Oncorhynchus contains eight species which occur naturally only in the North Pacific. These include Chinook salmon (Oncorhynchus tshawytscha), Chum salmon (Oncorhynchus keta), Coho salmon (Oncorhynchus kisutch), Masu salmon (Oncorhynchus masou), Pink salmon (Oncorhynchus gorbuscha) and Sockeye salmon (Oncorhynchus nerka). Most preferably, the fish is an Atlantic salmon (Salmo salar). The term “salmon” covers inter alia, salmonids.

The fish zygote is formed by fertilization of a fish oocyte. The zygote's genome is a combination of the DNA from the two gametes (oocyte and sperm). The zygote is at the one-cell stage, i.e. before cell division has started. Modification at this stage ensures that all cells in the fish will be modified in the same way (i.e. it avoids mosaicism).

In some embodiments of the invention (particularly those embodiments involving the modification of the embryonic genome), the embryo is an early-stage embryo, e.g. a 2-, 4- or 8-cell embryo, preferably a 2-cell embryo.

In some embodiments, the zygote, embryo or fish is male. In other embodiments, the zygote, embryo or fish is female.

As used herein, the term “germ cell survival factor gene” refers to genes whose elimination results in the absence of viable primordial germ cells (PGCs) in the fish (in the absence of the introduction of the protein or RNA encoded by the germ cell survival factor gene into the zygote). The term “germ cell survival factor gene” also refers to genes which are essential for the production of gametes or which are essential for the production of viable gonads.

Examples of such germ cell survival factor genes include those given in publications [9-12]. Preferably, the germ cell survival factor gene is one which is only present once in the haploid fish genome.

Preferably, the germ cell survival factor gene is a piwil-like gene or a piwi gene.

Piwi proteins within germ cells are known to associate with piwi-interacting RNAs (piRNAs), and subsequently suppress the expression of transposable elements. Consequently, the germline genome integrity is protected (Bao and Yan, 2012; Castaneda et al., 2011; Juliano et al., 2011). Mutations of piwi proteins, as shown in zebrafish with piwil1 mutations, cause progressive loss of germ cells, showing an important function of this protein for juvenile germ cell survival (Houwing et al., 2007). Searches in the salmon has revealed a single copy of the piwil1 gene (Kleppe et al 2015). In zebrafish, piwil1 is expressed in PGCs as well as adult germ cells (Draper et al., 2007; Houwing et al., 2007). Furthermore, piwil1 transcripts were maternally deposited in the egg and clearly present during the first cleavage stages until gastrulation. During the following developmental stages until hatching, piwil1 had a weaker expression (Zhao et al., 2012). Likewise we have shown that piwil1 expression is confined to germ cells of the testis and ovary in juvenile salmon; however with no expression in other tissues, suggesting a crucial role for piwil1 in gametogenesis in salmon (Kleppe et al 2015).

Most preferably, the germ cell survival factor gene is a piwi-like (piwil) gene, e.g. piwil1 or piwil2, or a paralog thereof.

Most preferably, the germ cell survival factor gene is piwil1, or a derivative or variant thereof.

Preferably, the piwil1 gene or the piwi gene is from Salmon, more preferably from Salmo salar (Atlantic salmon).

In one preferred embodiment, the piwil1 gene is gene ID 106585526 or an ortholog thereof. Orthologs may readily be identified from the NCBI database (www.ncbi.nlm.nih.gov/).

In some embodiments of the invention, the process comprises the step: (a) introducing protein or mRNA encoded by a germ cell survival factor gene, wherein the germ cell survival factor gene is a piwil gene or a piwi gene (e.g. piwil1), into a fish zygote or one or more cells of an early-stage fish embryo. The protein or RNA encoded by the germ cell survival factor gene may be introduced into the zygote or one or more cells (preferably all cells) of the early-stage fish embryo by any suitable method.

Examples of suitable methods include micro-injecting, electroporation, nano-particles and liposome delivery. Preferably, the protein or RNA encoded by the germ cell survival factor gene is introduced directly into the zygote or one or more cells (preferably all cells) of the early-stage fish embryo by micro-injection.

A functional non-wild-type amount of germ cell survival factor RNA or polypeptide is introduced or has previously been introduced into the fish zygote or early-stage fish embryo. The amount of RNA or protein which is introduced will be an amount which is sufficient to compensate for the loss of expression of the protein or mRNA encoded by the germ cell survival factor gene by the zygotic genome. The amount of mRNA or protein which is introduced will need to be an amount which provides sufficient RNA/protein to facilitate the normal migration of PGCs and the normal development of the gonads and gametes.

For example, in the early development of salmon, zygotic expression of the piwil1 gene is normally turned on at gastrulation. Consequently, the amount of piwil1 RNA or PIWIL1 protein which is introduced at the zygote stage will need to be sufficient to survive to the gastrulation stage and still be at a cellular concentration which is sufficient to facilitate PGC migration and gonadal development.

Preferably, the amount of the germ cell survival factor mRNA will be at least twice the amount of germ cell survival factor mRNA which is present in a corresponding wild-type (i.e. unmodified) zygote or cell (of the same fish species). In some embodiments, the amount of the germ cell survival factor mRNA is 0.1-20.0 ng mRNA, preferably 0.1-1.0, 1-10 or 10-20 ng per zygote or cell. In some embodiments, the amount of the germ cell survival factor mRNA is at least 0.1, 0.2, 0.3, 0.4 or 0.5 ng mRNA per zygote or cell. In other embodiments, the amount of the germ cell survival factor mRNA is at least 1, 2, 3, 4, 5, 6, 7, 8, 9 or 10 ng mRNA per zygote or cell.

Preferably, the amount of the germ cell survival factor protein will be at least twice the amount of germ cell survival factor protein which is present in a corresponding wild-type (i.e. unmodified) zygote or cell (of the same fish species).

In some embodiments, the amount of the germ cell survival factor protein (e.g. PIWILI) is 50-1000 pg per zygote or cell, preferably 200-800 or 300-600 pg per zygote or cell, more preferably about 400 pg per zygote or cell. In other embodiments, the amount of the germ cell survival factor protein (e.g. Piwil1) is 50-100, 100-200, 200-300, 300-400, 400-500, 500-600, 600-700, 700-800, 800-900 or 900-1000 pg per zygote or cell.

In some embodiments of the invention, the process comprises the step:
(a) modifying the genome of a fish zygote or the genome of one or more cells of an early-stage fish embryo to eliminate functional expression of a germ cell survival factor gene.

Preferably, the functional expression of both (if the genome is diploid) or all (if the genome is polyploid) copies of the germ cell survival factor gene are eliminated in the fish zygote in Step (a). Preferably, the functional expression of all copies of the germ cell survival factor gene are eliminated in all of the cells of the early-stage fish embryo in Step (a).

The genome of the fish zygote or early-stage fish embryo is modified to eliminate functional expression of or from the germ cell survival factor gene. As a consequence of this modification, viable primordial germ cells (PGCs) will not be produced in the fish (if the protein or RNA encoded by the germ cell survival factor gene is not introduced into the zygote or early-stage embryo, or at any later developmental stage).

As used herein, the term “eliminate functional expression” means that a functional or viable protein or RNA product of the germ cell survival factor gene is not produced. In some embodiments, a non-functional (e.g. mutated) mRNA or non-functional (e.g. mutated) protein product may be produced.

Similarly, the term “non-functional” as used herein in the context of a germ cell survival factor gene means that the copy or copies of the germ cell survival factor gene are not capable of producing a functional or viable protein or RNA product, and hence viable primordial germ cells (PGCs) will not be produced in the fish.

Equally, a non-functional gene-product, protein or polypeptide in the context of this invention is one which is non-efficacious. In the presence of such non-function gene-products, proteins or polypeptides (and in the absence of a corresponding functional gene-product, protein or polypeptide), viable primordial germ cells (PGCs) will not be produced in the fish.

The means to modify the genome of a fish zygote or early-stage fish embryo to eliminate expression of the germ cell survival factor gene may also be introduced into the zygote or early-stage fish embryo in a similar manner to that described above.

For example, the fish genome may be modified to introduce a change in one or more nucleotides within the germ cell survival factor gene. As used herein, the term “germ cell survival factor gene” includes its associated regulatory sequences (e.g. enhancers, promoters and terminators), i.e. not only the protein- or RNA-encoding sequences.

For example, the nucleotide sequence of the germ cell survival factor gene may comprise one or more additions, deletions or substitutions which result in the production of a non-functional (e.g. non-efficacious) germ cell survival factor gene product (e.g. RNA or protein). In some embodiments, the germ cell survival factor gene is wholly or partially deleted.

The nucleotide sequence may be modified in any suitable way. For example, the modification may be achieved using a CRISPR gRNA directed against the germ cell survival factor gene or its associated regulatory sequences, together with an appropriate endonuclease (e.g. Cas9, Cpf1). The introduction of a single or double-stranded break in the germ cell survival factor gene, followed by endogenous end-joining mechanisms may be sufficient to introduce a small (out of frame) deletion into the germ cell survival factor gene. Other means include the use of TALENs or zinc-finger proteins, which may be appropriately targeted against the germ cell survival factor gene.

Preferably, the modifying step comprises: introducing, into the fish zygote or early-stage fish embryo, a CRISPR gRNA directed against the germ cell survival factor gene, together with a Cas9 endonuclease or a nucleic acid encoding a Cas9 endonuclease, such that the CRISPR gRNA/Cas9 complex so formed creates a mutation in (one or more or all copies of) the germ cell survival factor gene rendering it or one of its gene products non-functional or non-viable.

The fish zygote genome will comprise both maternal and paternal chromosomes. It will therefore be bi-allelic (or multi-allelic) for the germ cell survival factor gene.

It is most preferred that both alleles (or all alleles in non-diploid fish) of the germ cell survival factor gene are modified in the fish zygote to eliminate all or substantially all functional genomic expression of the germ cell survival factor gene or its gene products.

In embodiments of the invention which relate to early-stage fish embryos, it is most preferred that all copies of the germ cell survival factor gene are modified to eliminate all or substantially all functional genomic expression of the germ cell survival factor gene or its gene products.

The genome of the fish zygote or fish embryo will be heritably-modified to eliminate functional expression of one or more or all copies of a germ cell survival factor gene, i.e. the modifications are ones which are transmissible to the progeny of the fish. In the context of this invention, therefore, the term “modifications” does not encompass the use of anti-sense RNA to make transient modifications. Hence genomes of the germ cells of the fish will not be capable of functional expression of the germ cell survival factor gene.

The means to modify the genome of a fish zygote or early-stage fish embryo to eliminate functional expression of one or more or all copies of the germ cell survival factor gene and the protein or RNA encoded by the germ cell survival factor gene (if desired) may be introduced into the zygote sequentially, simultaneously or separately.

The means to modify the genome of a fish zygote or early-stage fish embryo to eliminate functional expression of one or more or all copies of the germ cell survival factor gene may be introduced first and the protein or RNA encoded by the germ cell survival factor gene (if desired) may be introduced into the fish zygote or early-stage fish embryo second, or vice versa.

In some embodiments, the means to modify the genome of the fish zygote or early-stage fish embryo to eliminate functional expression of one or more or all copies of the germ cell survival factor gene is co-injected into the zygote (preferably at the one-cell stage) or early-stage fish embryo (preferably at the 2-cell stage) with the protein or RNA encoded by the germ cell survival factor gene.

Wild-type fish zygotes will contain a store of maternal germ cell survival factor RNA. This RNA provides sufficient germ cell survival factor protein to last at least until the time when the zygotic germ cell survival factor gene is turned on.

In contrast, the fish zygotes of the invention will comprise either significantly more germ cell survival factor RNA (F1 zygotes, as a consequence of the introduction of the RNA) compared to wild-type fish zygotes or they will contain no maternal or zygotically-expressed functional germ cell survival factor RNA (F2 zygotes, as consequence of the fact that the maternal germ cell survival factor gene or gene product is non-functional). Similar considerations apply to the early-stage fish embryos.

The invention therefore provides a fish zygote or early-stage fish embryo, wherein the fish zygote or early-stage fish embryo comprises a non-wild-type amount of a germ cell survival factor mRNA or protein.

As used herein, the term “non-wild type amount of a germ cell survival factor mRNA or protein” refers to an amount of germ cell survival factor mRNA or protein which is not present in wild-type zygotes or wild-type early-stage embryos from the species of fish in question.

In some embodiments, the fish zygote or early-stage embryo contains less than a wild-type amount of a germ cell survival factor mRNA or protein. For example, the fish zygote or early-stage fish embryo may contain 0-90% of the wild-type amount of germ cell survival factor mRNA or protein, preferably 0-50%, 0-20% or 0-10% of the wild-type amount of germ cell survival factor mRNA or protein.

In some preferred embodiments, the fish zygote or early-stage fish embryo comprises none or essentially none of the germ cell survival factor mRNA or protein.

In other embodiments, the fish zygote or early-stage fish embryo contains more than a wild-type amount of the germ cell survival factor mRNA or protein. For example, the fish zygote or early-stage fish embryo may contain 1.5-20× the wild-type amount of germ cell survival factor mRNA or protein, preferably 2-5×, 5-10× or 10-15× the wild-type amount of germ cell survival factor mRNA or protein.

In some embodiments, the fish zygote or early-stage fish embryo of the invention contains 0.1-10, preferably 0.1-1.0 or 1.0-10 ng of the germ cell survival factor mRNA. In some embodiments, the fish zygote or early-stage fish embryo contains about 0.1-0.2, 0.2-0.3, 0.3-0.4, 0.4-0.5, 0.5-0.6, 0.6-0.7, 0.7-0.8, 0.8-0.9 or 0.9-1.0 ng of the germ cell survival factor mRNA. In other embodiments, the fish zygote or early-stage fish embryo contains about 1-2, 2-3, 3-4, 4-5, 5-6, 6-7, 7-8, 8-9 or 9-10 ng of the germ cell survival factor mRNA.

A wild-type fish (e.g. salmon) zygote contains about 50 pg PIWIL1 protein per zygote. In some embodiments, the fish zygote or early-stage fish embryo of the invention comprises less than 200, preferably less than 150, 100, 90, 80, 70, 60, 50, 40, 30, 20 10 or 5 pg germ cell survival factor polypeptide (e.g. PIWIL1). In other embodiments, the fish zygote or early-stage fish embryo comprises more than 50, preferably more than 100, 200, 300, 400, 500, 600, 700, 800, 900 or 1000 pg germ cell survival factor polypeptide (e.g. PIWIL1). In some embodiments, the fish zygote or early-stage fish embryo comprises less than 500 or less than 1000 pg germ cell survival factor polypeptide (e.g. PIWIL1).

Preferably, the cell genomes of the fish zygote or early-stage fish embryo of the invention are not capable of expression of a functional variant of the germ cell survival factor gene.

The invention also provides a fish zygote or early-stage fish embryo, wherein the fish zygote or early-stage fish embryo comprises no or essentially no functional RNA or protein which is encoded by the germ cell survival factor gene, wherein the germ cell survival factor gene is a piwil gene or a piwi gene (e.g. piwil1).

A further aspect of the invention relates to broodstock (F1) fish and processes for their production. Broodstock fish may be produced from the fish zygotes or early-stage fish embryos of the invention.

The cells of the broodstock (F1) zygotes, embryos and fish may be mosaic for mutations in the germ cell survival factor genes due to the fact that targeted mutations generally do not occur in the first cell stage in fish (e.g. salmon) embryos injected with Crispr-Cas9 mutational complexes. The mutations occur in subsequent cells formed after the first cell divison in the embryo (e.g. Edvardsen et al., 2014). Clearly, if differential genome modifications are introduced into mulltiple cells of an early-stage fish embryo, the cell genomes of those embryos will inevitably be mosaic for mutations in the germ cell survival factor genes.

The invention therefore also provides a process for producing a broodstock fish, the process comprising the steps:
(a)(i) culturing a fish zygote or early-stage fish embryo of the invention, or
(a)(ii) producing a modified fish zygote or early-stage fish embryo by a process for producing a modified fish zygote or early-stage fish embryo of the invention and culturing the fish zygote or early-stage fish embryo to produce a cultured fish; and
(b) growing the cultured fish to produce a juvenile broodstock fish, and optionally
(c) growing the juvenile broodstock fish to produce a sexually-mature broodstock fish.

As used herein, the term “culturing” a fish zygote or early-stage fish embryo refers to the process of allowing or facilitating the fish zygote or early-stage fish embryo to develop to form a multi-cellular organism (e.g. a salmonid).

Similarly, the term “growing” as used herein refers to the process of feeding the fish and allowing it to grow to form a juvenile fish, an adult fish or a sexually-mature fish.

The broodstock fish are not capable of producing functional germ cell survival factor genes or gene-products, due to the mutations in their germ cell survival factor genes. These fish will have gonads which are capable of producing viable sperm or eggs, due to presence of the RNA or protein of the germ cell survival factor gene which was present or introduced at the zygote or early-stage embryo stage.

The invention therefore also provides a juvenile or sexually-mature (broodstock) fish:
(a) whose cells collectively comprise one or more (preferably 5-15) genomic mutations in a germ cell survival factor gene, wherein the germ cell survival factor gene is a piwil gene or a piwi gene (e.g. piwil1), wherein the one or more mutations render one or more or all (preferably all) copies of the germ cell survival factor gene or gene-product non-functional; and
(b) which has gonads which are capable of producing viable sperm or eggs.

The cells of the above-mentioned zygotes, embryos, juvenile or sexually-mature (F1, broodstock) fish will generally be mosaic for mutations in the germ cell survival factor gene (preferably piwil1) for the reasons discussed above. Overall, the population of cells in any one such fish may collectively comprise 3-20, more preferably 5-15, different mutations in the germ cell survival factor gene which render one or more or all (preferably all) copies of the germ cell survival factor gene or gene-product non-functional. Any one cell in this population of cells will, however, only have 1-2 such mutations.

As used herein, the term “collectively” means in total.

The broodstock (F1) fish of the invention are fertile and hence they are capable of producing gametes, i.e. sperm or eggs. In contrast to the cells of the broodstock fish (which collectively will be mosaic for mutations in the germ cell survival factor gene), the gametes of the broodstock fish will not be mosaic (since they only contain one haploid genome).

The invention therefore also provides sperm or eggs (oocytes) from a sexually-mature (broodstock) fish of the invention.

In particular, the invention provides a fish oocyte:
(a) which comprises no or essentially no functional RNA or protein which is encoded by the germ cell survival factor gene, wherein the germ cell survival factor gene is a piwil gene or a piwi gene (e.g. piwil1); and/or
(b) whose genome comprises one or more mutations (preferably 1-2) in a germ cell survival factor gene, wherein the germ cell survival factor gene is a piwil gene or a piwi gene (e.g. piwil1), wherein the one or more mutations render one or more or all (preferably all) copies of the germ cell survival factor gene or gene-product non-functional.

The invention also provides a fish sperm whose genome comprises one or more (preferably 1-2) mutations in a germ cell survival factor gene, wherein the germ cell survival factor gene is a piwil gene or a piwi gene (e.g. piwil1), wherein the one or more mutations render one or more or all (preferably all) copies of the germ cell survival factor gene or gene-product non-functional.

In particular, the invention provides a salmon:
(a) whose cells collectively comprise one or more (preferably 5-15) genomic mutations in the piwil1 gene, wherein the one or more mutations render one or more or all (preferably all) copies of the piwil1 gene or PIWIL1 protein non-functional; and
(b) which has gonads which are capable of producing viable sperm or eggs.

In this embodiment, the eggs and sperm will be viable but, due to the presence of the bi-allelic knockout of the piwil1 gene in the haploid genomes of the eggs and sperm, all off-spring of such salmon will lack germ cells. Hence all such off-spring will be sterile.

One key aim of the invention is to provide a plurality of fish (i.e. F2 farmed fish) which are incapable of breeding with wild-type fish if they escape from their breeding areas, i.e. which are sterile. This is achieved by the processes described herein whereby a germ cell survival factor gene or corresponding gene-product which is normally required for proper gonadal development is rendered non-functional (or deleted).

Female broodstock fish of the invention (or oocytes obtained therefrom) may be crossed either with male broodstock fish of the invention or wild-type fish (or sperm obtained therefrom) to produce F2 zygotes. Due to the absence of functional germ cell survival factor genes or corresponding gene-products in the female broodstock fish, the oocytes which are produced by such fish will not contain functional germ cell survival factor RNA or protein. Consequently, fish derived from such oocytes will be sterile.

The F2 fish of the invention are sterile/infertile because they have no germ cells and they are therefore not capable of producing gametes. In contrast to the cells of the broodstock fish (which will be mosaic for mutations in the germ cell survival factor gene), the cells of the F2 fish will be significantly less mosaic because those cells will have been derived from two haploid genomes (either from two genomes which have no functional germ cell survival factor genes (e.g. from crossing two F1 fish) or from one genome which has no functional germ cell survival factor genes (e.g. an F1 fish) and one wild-type fish). Generally, the cells of these F2 fish will collectively only have 1-2 mutations in their genomes; these mutations will render one or more or all copies of the germ cell survival factor gene (preferably piwil1) non-functional. Some cells in these F2 fish will have one mutation; other cells will have a different mutation.

In a preferred embodiment, therefore, the invention provides a fish zygote:
(a) wherein the genome of the fish zygote comprises one or two mutations which render one or more or all copies of the germ cell survival factor gene non-functional, wherein the germ cell survival factor gene is a piwil gene or a piwi gene (e.g. piwil1; and
(b) wherein the zygote does not comprise functional RNA or functional protein encoded by the germ cell survival factor gene (preferably piwil1).

The invention particularly relates to embryos and fish which have developed from such zygotes. Such F2 fish will be sterile (due to the absence of PGCs). These F2 fish can be farmed in the vicinity of wild-type fish in the knowledge that the F2 fish cannot interbreed with wild-type fish.

In a further embodiment, the invention provides a sterile fish (preferably a salmon):
(a) whose cells collectively comprises one or more (preferably only 1-2) different mutations in their genomes which render one or more or all copies of a germ cell survival factor gene non-functional, wherein the germ cell survival factor gene is a piwil gene or a piwi gene (e.g. piwil1); and/or
(b) wherein the physiological and/or anatomical features of the fish are characteristic of a fish that has developed from a zygote which was lacking in maternally-derived mRNA encoded by the germ cell survival factor gene.

In some embodiments, all of the cells of the fish comprise a first specific mutation in their genomes which renders one or more or all copies of the germ cell survival factor gene (preferably piwil1) non-functional.

Preferably, the pattern of germ cell survival factor gene mutations is uniform (i.e. not mosaic) within all of the gonadal cells of the fish. Preferably, the pattern of germ cell survival factor gene mutations is uniform (i.e. not mosaic) within all of the cells of the fish.

In some other embodiments, a first population of cells of the fish comprise a first specific mutation in their genomes which renders one or more or all copies of the germ cell survival factor gene (preferably piwil1) in those cells non-functional, and a second population of cells (or the remaining cells) of the fish comprise a second (different) specific mutation in their genomes which renders one or more or all copies of the germ cell survival factor gene (preferably piwil1) in those cells non-functional.

Preferably, the sterile fish has one or more of the following:
(i) no germ cells;
(ii) testes or ovaries without germ cells;
(iii) testicular spermatogenic tubules without germ cells (male fish); and
(iv) gonads which lack ovarian follicles (female fish).

Preferably, the fish is an adult fish (e.g. older than 6 months, 12 months, 24 months or 36 months).

Preferably, the zygote was one which lacked any (endogenous or exogenous) mRNA or protein encoded by the germ cell survival factor gene.

Preferably, the physiological and/or anatomical features are features of the fish's reproductive system, e.g. its gonads.

The disclosure of each reference set forth herein is specifically incorporated herein by reference in its entirety.

### BRIEF DESCRIPTION OF THE FIGURES

FIG. 1. Gross morphology (A and C) and section of gonad (E) of a rescued dndKO female (VIRGIN female) with germ cells produced by transient injection of dnd mRNA into the zygote. Gross morphology (B and D) and section of gonad (F) of a dndKO female.

FIG. 2. Gross morphology (A and D) and section of gonad (G) of a control immature male. Gross morphology (B and E) and section of gonad obtained from a rescued dndKO male (VIRGIN male) with germ cells (H) produced by transient injection of dnd mRNA into the zygote. Gross morphology (C and F) and section of a dndKO male gonad (I), lacking germ cells and containing numerous Sertoli cells.

FIG. 3. Expression of vasa (a germ cell specific marker) in gonads obtained from one year old control immature fish, dndKO fish and dndKO rescued fish. dndKO fish were produced by transient injection of dnd mRNA into the zygote. A and B illustrate expression of vasa in gonads obtained from females and males, respectively.

FIG. 4. Mutational analysis of fin clips obtained from the dnd knockout (dndKO) fish, control and dndKO rescued fish produced by transient injection of dnd mRNA into the zygote. The top sequence is the genomic region of dnd and below is the target gRNA. This is followed by the dndKO and dndKO rescued animals and at the bottom wildtype sequences for dnd in male and female control are shown. (SEQ ID NOs: 3-9; some sequences are repeated.)

FIG. 5. Deep sequencing of dnd CRISPR target site, using DNA obtained from fin clips of wt, germ cell free (GCF) and rescued males and females. Each fin clip were sequenced to a depth ranging between 50,000-400,000.

FIG. 6. Histology and gross morphology of a control (A and C) and rescued maturing male with 100% dnd mutation rate (B and D). The rescued male displayed normal gross morphology and histology, and showed the characteristic spermatogonial stages.

FIGS. 7A and 7B. Histology of piwil1^{-/-} and piwil1^{+/+} immature salmon testis.

FIGS. 8A and 8B: Histology of piwil1^{-/-} and piwil1^{+/+} immature salmon ovary.

### EXAMPLES

Examples 1-4 are not examples of the invention. They are provided for enablement purposes in order to demonstrate how the invention may be worked using a diffferent germ cell survival factor gene, i.e. dead end (dnd).

### Example 1

### Materials and Methods

Preparation of Salmon Zygotes

Salmon eggs and sperm were obtained from Aquagen (Trondheim, Norway). These were sent overnight to Matre Aquaculture station, Norway. Eggs were subsequently fertilized with sperm in fresh water (6-8° C.) containing 0.5 mM reduced gluthathione as described for rainbow trout [13]. After fertilization, embryos were incubated 2-3 hours at 6-8° C. until the first cell was visible.

Preparation of CRISPR sgRNA and dnd RNA

BamHI-HF (NEB) linearized pT7-gRNAs including the respective cloned target sites were cleaned up using a QIAprep column (Qiagen) and transcribed using the MEGAscript T7 kit (Ambion) according to the manufacturer's protocol. The mirVana miRNA Isoltation Kit was used to purify gRNAs.

For producing Cas9 nuclease mRNA, we used the pTST3-nCas9n vector optimized for Zebrafish (Jao et al., 2013; Addgene ID #46757). Prior to in-vitro transcription, the plasmid was linearized using XbaI (NEB) and cleaned up via a QIAprep Spin column. Cas9 mRNA was produced using the mMessage mMachine T3 kit (Ambion) and purified using an RNeasy MiniKit spin column (Qiagen).

Full length dnd mRNA was PCR amplified from salmon ovary using q5 polymerase, using a forward primer with T7 attached to it. The PCR product was gel-purified (Qiagen gel purification kit) and sequenced. The dnd PCR product was in vitro transcribed into a functional dnd mRNA using T7 ARCA mRNA kit (NEB).

Micro-Injection of CRISPR sgRNA and dnd RNA into Zygotes

Eggs were micro-injected with 2-8 nl of a mix containing 50 ng/ml gRNA, 100 ng/ml mRNA for dnd and 150 ng/ml Cas9 mRNA in MilliQ H₂O using the picospritzer III (Parker Automation, UK) and needles from Narishige (Japan). After injection, eggs were incubated at 6° C. until hatching.

Testing for the Results Using Fin Clips

DNA was obtained from embryos, juveniles and fin clips using DNeasy Blood & Tissue kit (Qiagen) or AllPrep DNA/RNA kit (Qiagen) with the following modifications: Juveniles (separated from the yolk sac) and fin clips were homogenized using Zirconium oxide beads and a homogenizer (Precellys) in buffer ATL or buffer RLTplus/β-mercaptoethanol prior to DNA extraction. PCR was performed on genomic DNA to obtain a fragment that covered the targeted mutagenesis site [7]. Fragments were both directly sequenced, and sub-cloned into pCR4-TOPO using the TOPO TA cloning kit for sequencing (Invitrogen) to either measure the general effect in the target site in the whole preparation or in single sequences from clones to assess the level of mutation rate in each individual or sample.

### Example 2

### Production of Broodstock Fish

To establish a dnd KO stable broodstock line, FO fish were obtained following the methods given in Example 1. Essentially, salmon zygotes were micro-injected with a gRNA (SEQ ID NO: 1) which targeted dnd and CRISPR Cas9 together with mRNA (SEQ ID NO: 2) coding for Dnd.

The gRNA sequence was:
(SEQ ID NO: 1)
5′-GGGCCCACGGCACGGAACAGCGG-3′.
mRNA sequence for Dnd
>JN712911.1 Salmo salar Dead end mRNA, complete cds
(SEQ ID NO: 2)
GAAAGTTGCTACTTTTTCGAGACCTAGGATAATGGAGGAGCGTTCAAGTCAGGTGTTGAACCCGGAGCGA
CTGAAGGCGCTGGAGATGTGGCTGCAGGAGACTGACGTCAAACTGACCCAGGTCAATGGCCAGAGGAAAT
ATGGAGGTCCACCTGATGACTGGCTTGGCGCCCCCCCTGGGCCGGGCTGTGAGGTGTTCATCAGCCAGAT
CCCGCGGGATGTCTTTGAGGACCAGCTGATTCCGCTGTTCCGTGCCGTGGGCCCTCTCTGGGAGTTCCGC
CTCATGATGAACTTCAGCGGACAGAACCGTGGCTTTGCCTACGCCAAGTACGACAGCCCTGCCTCGGCCG
CTGCCGCCATCCGCTCGTTGCATGGCCGTGCCCTCGAGTCAGGGGCACGCCTCGGTGTACGGCGCAGCAC
GGAGAAACGTCAGCTCTGTCTTGGGGAGCTGCCCACCAGCACAAGGAGGGAGCAACTGCTGCAGGTGCTG
CTGGACTTCTCTGAGGGGGTAGAGGGCGTGTCCCTGAGAGCAGGGCCTGGGGAACAGGGGATGTCTGCAG
TGGTGGTCTATGCCTCCCACCATGCAGCTTCCATGGCCAAGAAGGTGCTGATTGAAGCCTTTAAAAAACG
CTTCGGGCTGGCCATCACTTTGAAGTGGCAGTCCTCTTCTAGGCCCAAGCACGAAGAGCCTCCCAGACCC
TCCAAAACCCCTCCTTCCTCTCCTCCCAAACCTCCTCGCTGCTCCCTCCTGGACAGCCCCCGGCCTCCCC
TGCACCTCGCCCAGCGTCAGCTCCCTGCCTTCTCCCGGGCTGTGAGGGCGCCCTCTCCCATGGTGCACGC
TGCTCCTGAATCCCCCAGGGGGGCGACCATGGTGCCTCCTGTGGATGCAGCAGCCCTGCTCCAGGGTGTG
TGTGAGGTGTACGGGCAGGGGAAGCCCCTCTATGACCTGCAGTACCGCCACATGGGGCCTGACGGGTTCC
TGTGCTTCAGCTACCGGGTGTATGTGCCGGGGCTGGCCACACCCTTCACTGGGATGGTGCAGACTCTGCC
CGGCCCCACCCCTGGAGCCATACAGGAAGAGGCTCGCAGAGCTACAGCCCAGCAGGTCCTCAGCGCTCTG
TACAGGGCCTGATGGTGTTGAAGCACAGATCCCCTACTTTGTTTTAATTATGAAAATACTTAAATGTTTT
GCACTCTTTTATATTTAGTAAGTAGATGCATGATTTTACTTTTTTTTTTGAACCACTTTTGCATGTTTCT
GCACCATTTAATTGTTTCTCATTATAATAAAATGAGATTTGTCAAAAAAAAAAAAAAAAAAAAAAA

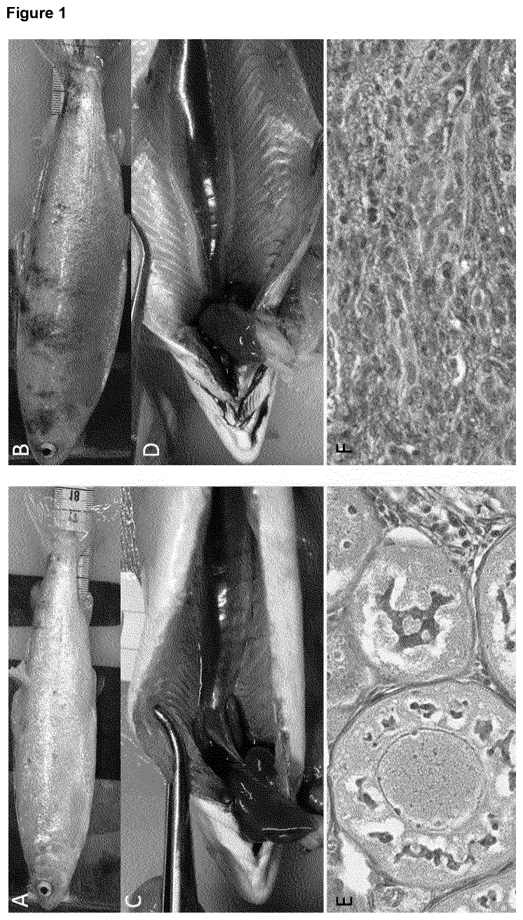

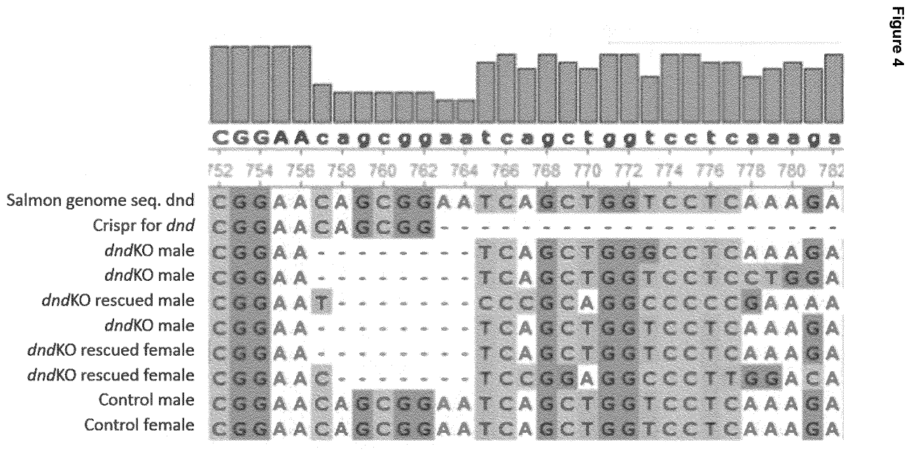

The fish were grown to a size suitable for pit-tag and fin-clip e.g. 10-15 g. DNA was extracted fom fin clips, to be able to determine if fish were mutated in the dnd gene (FIGS. 4 and 5). Fish with mutations in; the dnd gene, mutations in the dnd gene+mRNA for dnd and control, were sampled for gonad gross morphology, histology and gene expression in ˜25 g fish (FIGS. 1, 2 and 3).

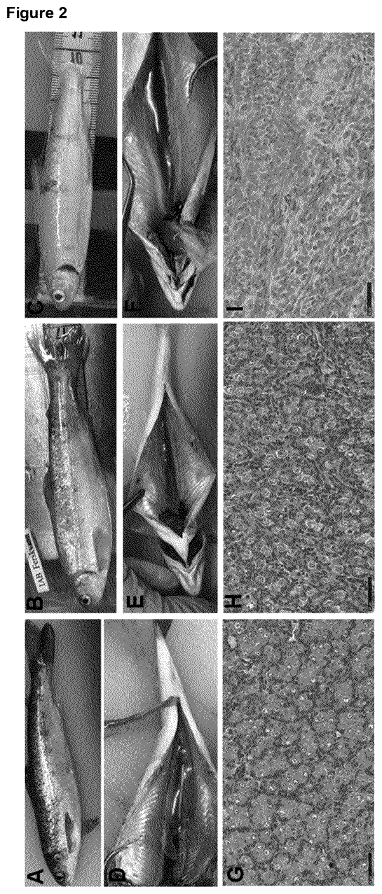

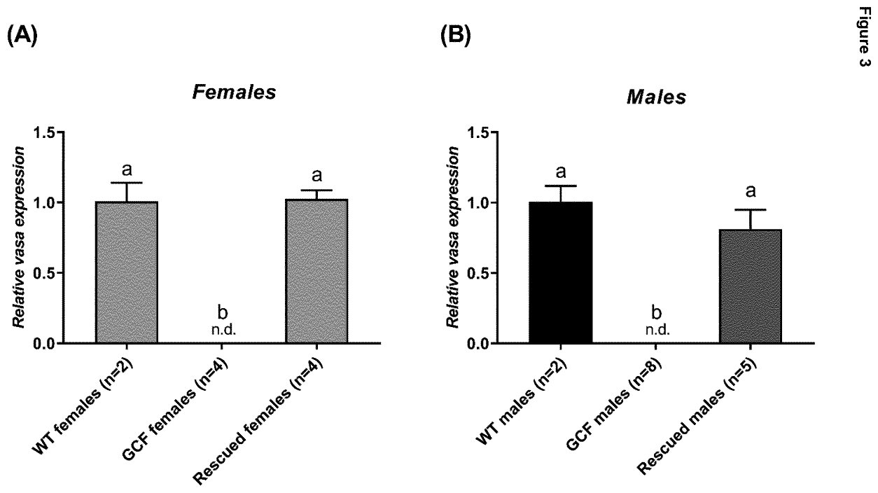

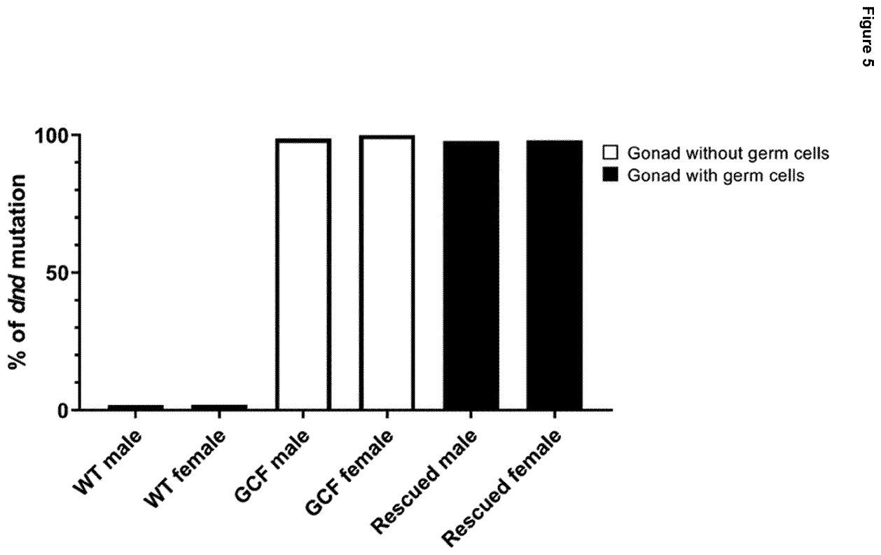

As shown in FIGS. 4 and 5, the rescued fish had mutations in the dnd gene, while at the same time having germ cells (FIGS. 1 and 2) and expressing the germ cell marker vasa (FIG. 3). The results demonstrate that it is possible to produce fish with germ cells from a fish with double allelic mutations in the dnd gene (FIG. 5). The results also show that dnd is not essential for further development of germ cells beyond the embryonic stage up to 2.5 years of age. We have also observed that dnd-rescued males can enter into puberty (FIG. 6). Dnd is therefore a suitable target as a germ cell survival factor and is not necessary for normal puberty in males (FIG. 6).

### Example 3

### Production of Farmed Fish

Gametes from the broodstock fish produced in Example 2 are used to produce salmon zygotes which have dnd biallelic knockouts. The fish which result from these zygotes have no PGCs and hence are sterile.

Each broodstock female can produce between 5,000-10,000 eggs and males can fertilize an immense number of eggs. The salmonids are used for farming and at the juvenile stage they are sampled to confirm lack of germ cells. The genomes of some individuals are sequenced to exclude fish with off-target mutations and to fully characterize the broodstock mutation.

### Example 4

### Production of Further Broodstock Fish

Gametes from the broodstock fish produced in Example 2 are used to produce salmon zygotes which have dnd biallelic mutations.

These zygotes are micro-injected with 0.2-0.5 ng of mRNA coding for dnd, in order to produce further broodstock fish (having viable PGCs and capable of producing gametes).

These “rescued” F1 broodstock fish are grown to a size suitable for pit-tag and fin-clip, and the specific mutations are characterized by sequencing of fin clips. Some of the fish are histologically and molecularly characterised in order to ensure that the rescue effect is successful.

### Example 5

### Production of Piwil1 Knockout (KO) Salmon Embryos

To elucidate the function of piwil1 in salmon, we knocked-out the piwil1 gene in salmon using CRISPR-Cas9. We detected a high mutation rate in F0 and the histology of gonads of piwil1 KO mutants was evaluated in comparison to controls. In F0, no apparent differences between controls and mutants were detected. In fact, in histological sections from immature, maturing and mature gonads from salmon piwil1 KO, no irregular phenotypes were detected. Also, at maturation in both sexes, no apparent reduction in the number of mature animals was detected for each sex: for males, 100% (n=11) of the control and 87.5% of piwil1KO (n=16); while for females 34% of piwil1KO females (n=26) and 45% of control females (n=15).

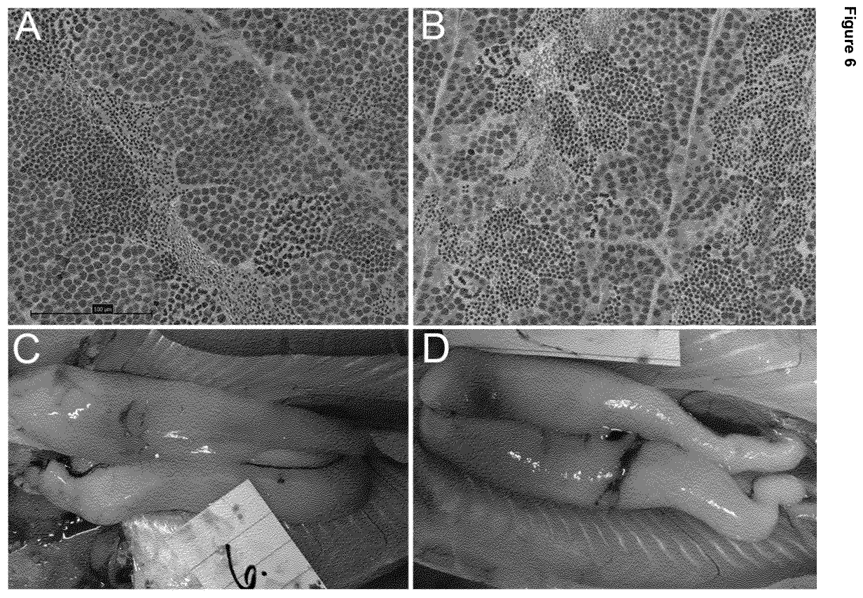

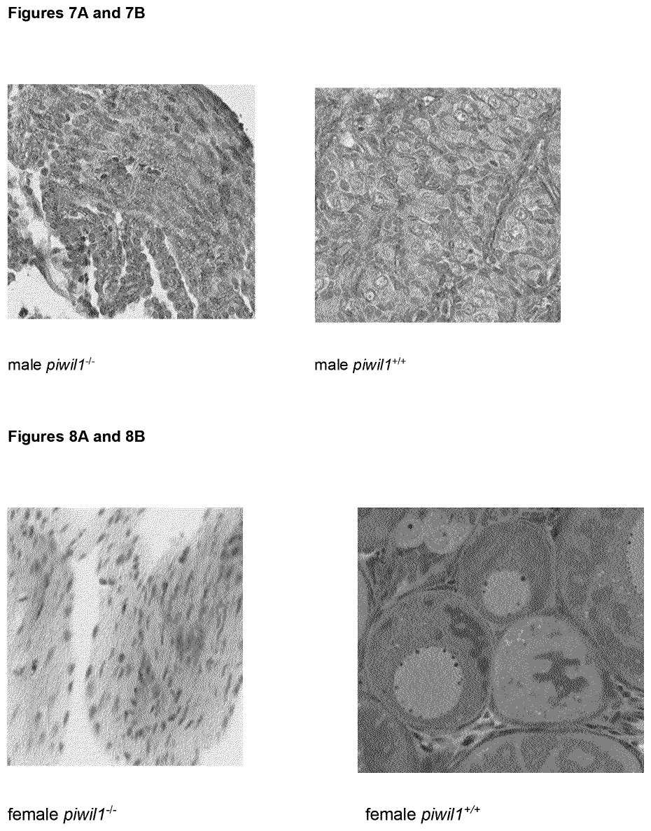

To elucidate a potential effect in the F1 generation, we intercrossed four piwil1 KO fish: 2 males and 2 females. At one year of age, we opened fish of both sexes which were either piwil1^{-/-}, piwil1^{-/+} and piwil1^{+/+}. The phenotype was evaluated with histology and genotyped with Sanger sequencing. All piwil1^{+/+} fish displayed normal germ cells in both sexes (FIGS. 7B and 8B). Both males and females displaying the piwil1^{-/-} genotype were germ-cell free (FIGS. 7A and 8A).

These results indicate that piwil1 is only essential for early primordial germ cell formation in salmon, while the adult and juvenile expression of this gene is non-essential for a normal reproductive path in both sexes. The piwil1 transcript therefore represents a highly usable transcript for rescue of germ cells in Atlantic salmon as the function of this protein is only important for formation of primordial germ cells.

### Example 6

### Production of Broodstock Fish

To establish a piwil1 KO stable broodstock line, F0 fish are obtained following the methods given in Examples 1-2, but using piwil1 genes. Essentially, salmon zygotes are micro-injected with a gRNA which targets piwil1 and CRISPR Cas9. In contrast to dnd, it may not be necessary to rescue the salmon zygotes by the co-injection of piwil1 mRNA because the amount of maternal piwil1 mRNA in the wild-type zygotes may be sufficient on its own (without zygotic expression of piwil1) to enable to production of viable gametes.

The fish are grown to a size suitable for pit-tag and fin-clip, e.g. 10-15 g. DNA is extracted from fin clips to be able to determine whether the fish are mutated in the piwil1 gene (in the same manner as in Example 2). Fish with mutations in the piwil1 gene, mutations in the piwil1 gene+mRNA for piwil1 and control, are sampled for gonad gross morphology, histology and gene expression in ˜25 g fish.

The rescued fish are expected to be while at the same time having germ cells and expressing the germ cell marker vasa. The results demonstrate that it is possible to produce fish with germ cells from a fish with double allelic mutations in the piwil1 gene.

### Example 7

### Production of Farmed Fish and Further Broodstock Fish

Farmed fish and broodstock fish which have piwil1 biallelic knockouts are produced as described in Example 3, using the piwil1 gene/mRNA instead of the dnd gene/mRNA.

Salmon zygotes which have piwil1 biallelic mutations are produced as described in Example 4 using the piwil1 gene/mRNA instread of the dnd gene/mRNA.

### REFERENCES

1. Taranger G L, Karlsen O, Bannister R J, Glover K A, Husa V, Karlsbakk E, Kvamme B O, Boxaspen K K, Bjorn P A, Finstad B et al: Risk assessment of the environmental impact of Norwegian Atlantic salmon farming. Ices J Mar Sci 2015, 72(3):997-1021.

2. Sambroni E, Abdennebi-Najar L, Remy J J, Le Gac F: Delayed sexual maturation through gonadotropin receptor vaccination in the rainbow trout Oncorhynchus mykiss. General and comparative endocrinology 2009, 164(2-3):107-116.

3. Wong T T, Zohar Y: Production of reproductively sterile fish: A mini-review of germ cell elimination technologies. General and comparative endocrinology 2015, 221:3-8.

4. Bedell V M, Westcot S E, Ekker S C: Lessons from morpholino-based screening in zebrafish. Briefings in functional genomics 2011, 10(4):181-188.

5. Fjelldal P G, Hansen T: Vertebral deformities in triploid Atlantic salmon (Salmo salar L.) underyearling smolts. Aquaculture 2010, 309(1-4):131-136.

6. Zohar Y, Munoz-Cueto J A, Elizur A, Kah O: Neuroendocrinology of reproduction in teleost fish. General and comparative endocrinology 2010, 165(3):438-455.

7. Wargelius A, Leininger S, Skaftnesmo K O, Kleppe L, Andersson E, Taranger G L, Schulz R W, Edvardsen R B: Dnd knockout ablates germ cells and demonstrates germ cell independent sex differentiation in Atlantic salmon. Scientific reports 2016, 6:21284.

8. Kleppe L, Andersson E, Skaftnesmo K O, Edvardsen R B, Fjelldal P G, Norberg B, Bogerd J, Schulz R W, Wargelius A: Sex steroid production associated with puberty is absent in germ cell-free salmon. Scientific reports 2017, 7(1):12584.

9. Kleppe L, Edvardsen R B, Furmanek T, Andersson E, Juanchich A, Wargelius A: bmp15l, figla, smc1bl, and larp6l are preferentially expressed in germ cells in Atlantic salmon (Salmo salar L.). Molecular reproduction and development 2017, 84(1):76-87.

10. Kleppe L, Wargelius A, Johnsen H, Andersson E, Edvardsen R B: Gonad specific genes in Atlantic salmon (Salmon salar L.): characterization of tdrd7-2, dazl-2, piwil1 and tdrd1 genes. Gene 2015, 560(2):217-225.

11. Nagasawa K, Fernandes J M, Yoshizaki G, Miwa M, Babiak I: Identification and migration of primordial germ cells in Atlantic salmon, Salmo salar: characterization of vasa, dead end, and lymphocyte antigen 75 genes. Molecular reproduction and development 2013, 80(2):118-131.

12. Koprunner M, Thisse C, Thisse B, Raz E: A zebrafish nanos-related gene is essential for the development of primordial germ cells. Genes & development 2001, 15(21):2877-2885.

13. Yoshizaki G, Takeuchi Y, Sakatani S, Takeuchi T: Germ cell-specific expression of green fluorescent protein in transgenic rainbow trout under control of the rainbow trout vasa-like gene promoter. The International journal of developmental biology 2000, 44(3):323-326.

14. Zhang Y, Chen J, Cui X, Luo D, Xia H, Dai J, Zhu Z, Hu W A controllable on-off strategy for the reproductive containment of fish. Sci Rep. 2015 Jan. 5; 5:7614

15. Noguchi T, Noguchi M. J A recessive mutation (ter) causing germ cell deficiency and a high incidence of congenital testicular teratomas in 129/Sv-ter mice. Natl Cancer Inst. 1985 August; 75(2):385-92.

16. Youngren K K, Coveney D, Peng X, Bhattacharya C, Schmidt L S, Nickerson M L, Lamb B T, Deng J M, Behringer R R, Capel B, Rubin E M, Nadeau J H, Matin A. The Ter mutation in the dead end gene causes germ cell loss and testicular germ cell tumours. Nature. 2005 May 19; 435(7040):360-4.

17. Northrup E, Zschemisch N H, Eisenblätter R, Glage S, Wedekind D, Cuppen E, Dorsch M, Hedrich H J. The ter mutation in the rat Dnd1 gene initiates gonadal teratomas and infertility in both genders. PLoS One. 2012; 7(5): e38001.

18. Zechel J L, Doerner S K, Lager A, Tesar P J, Heaney J D, Nadeau J H. Contrasting effects of Deadend1 (Dnd1) gain and loss of function mutations on allelic inheritance, testicular cancer, and intestinal polyposis. BMC Genet. 2013 Jun. 17; 14:54

19. Houwing S, Berezikov E, Ketting R F. Zili is required for germ cell differentiation and meiosis in zebrafish. EMBO J. 2008 Oct. 22; 27(20):2702-11. doi: 10.1038/emboj.2008.204. Epub 2008 Oct. 2. PMID:18833190

20. Houwing S, Kamminga L M, Berezikov E, Cronembold D, Girard A, van den Elst H, Filippov D V, Blaser H, Raz E, Moens C B, Plasterk R H, Hannon G J, Draper B W, Ketting R F. A role for Piwi and piRNAs in germ cell maintenance and transposon silencing in Zebrafish. Cell. 2007 Apr. 6; 129(1):69-82. PMID:17418787

21. Bao J, Wang L, Lei J, Hu Y, Liu Y, Shen H, Yan W, Xu C. STK31(TDRD8) is dynamically regulated throughout mouse spermatogenesis and interacts with MIWI protein. Histochem Cell Biol. 2012 March; 137(3):377-89. doi: 10.1007/s00418-011-0897-9. Epub 2011 Dec. 29. PMID: 22205278

22. Castañeda J, Genzor P, Bortvin A. piRNAs, transposon silencing, and germline genome integrity. Mutat Res. 2011 Sep. 1; 714(1-2):95-104. doi: 10.1016/j.mrfmmm.2011.05.002. Epub 2011 May 11. Review. PMID: 21600904

23. Uniting germline and stem cells: the function of Piwi proteins and the piRNA pathway in diverse organisms.

24. Juliano C, Wang J, Lin H. Annu Rev Genet. 2011; 45:447-69. doi: 10.1146/annurev-genet-110410-132541. Epub 2011 Sep. 19. Review. PMID: 21942366 Specific expression of Olpiwi1 and Olpiwi2 in medaka (Oryzias latipes) germ cells.

25. Zhao H, Duan J, Cheng N, Nagahama Y. Biochem Biophys Res Commun. 2012 Feb. 24; 418(4):592-7. doi: 10.1016/j.bbrc.2011.12.062. Epub 2011 Dec. 20. PMID: 22209791

### SEQUENCE LISTING FREE TEXT

<210> 1 <223> gRNA sequence

1.-6. (canceled)

7. A juvenile or sexually-mature fish:
(a) whose cell genomes collectively comprise one or more mutations in a germ cell survival factor gene, wherein the one or more mutations render all copies of the germ cell survival factor gene or gene product in the fish non-functional, wherein the germ cell survival factor gene is a piwil gene or a piwi gene; and
(b) which has gonads which are capable of producing viable sperm or eggs.

8. Sperm or eggs from the sexually-mature fish as claimed in claim 7.

9. A fish zygote:
(a) whose genome comprises one or more mutations which render one or more or all copies of a germ cell survival factor gene non-functional,
wherein the germ cell survival factor gene is a piwil gene or a piwi gene; and
(b) wherein the zygote does not comprise functional RNA or functional protein encoded by the germ cell survival factor gene.

10. (canceled)

11. A sterile fish:
(a) whose cell genomes collectively comprise one or more mutations which render one or more or all copies of a germ cell survival factor gene in the fish non-functional,
wherein the germ cell survival factor gene is a piwil gene or a piwi gene; and
(b) wherein the physiological and/or anatomical features of the fish are characteristic of a fish that has developed from a zygote which was lacking in maternally-derived mRNA encoded by the germ cell survival factor gene.

12. The sterile fish of claim 11, wherein the fish has:
(i) no germ cells;
(ii) testes or ovaries without germ cells;
(iii) testicular spermatogenic tubules without germ cells; or
(iv) gonads which lack ovarian follicles.

13.-16. (canceled)

17. The juvenile or sexually-mature fish of claim 7:
(a) whose cell genomes collectively comprise from 3-20 mutations in the germ cell survival factor gene, wherein the 3-20 mutations render all copies of the germ cell survival factor gene or gene product in the fish non-functional.

18. The fish zygote of claim 9:
(a) whose genome comprises 1-2 mutations which render one or more or all copies of the germ cell survival factor gene non-functional.

19. The sterile fish of claim 11:
(a) whose cell genomes collectively comprise 1-2 mutations which render one or more or all copies of the germ cell survival factor gene in the fish non-functional.

20. The juvenile or sexually-mature fish of claim 7, wherein the fish is from the family Salmonidae or the fish is a salmon.

21. The fish zygote of claim 9, wherein the fish is from the family Salmonidae or the fish is a salmon.

22. The sterile fish of claim 11, wherein the fish is from the family Salmonidae or the fish is a salmon.

23. The juvenile or sexually-mature fish of claim 7, wherein the germ cell survival factor gene is a piwil1 gene.

24. The fish zygote of claim 9, wherein the germ cell survival factor gene is a piwil1 gene

25. The sterile fish of claim 11, wherein the germ cell survival factor gene is a piwil1 gene.
